(19) United States
(12) Patent Application Publication
(10) Pub. No.: US 2003/0181327 A1
(43) Pub. Date: Sep. 25, 2003

(54) Microencapsulated magnetite support for cobalt fischer-tropsch catalyst

(75) Inventors: Joe D. Allison, Ponca City, OK (US); Yaming Jin, Ponca City, OK (US); Thomas D. Baugh, Ponca City, OK (US)

Correspondence Address: DAVID W. WESTPHAL, CONOCOPHILLIPS COMPNAY, P.O. BOX 1267, PONCA CITY, OK, 74602-1267, US

(73) Assignee: Conoco Inc., Houston, TX (US)

(21) Appl. No.: 10/100,966

(22) Filed: Mar. 19, 2002

(51) Int. Cl. B01J 23/00; C07C 27/06

(52) U.S. Cl. 502/325; 518/719

(57) ABSTRACT

Catalysts with silica-encapsulated magnetic supports are disclosed, along with their manner of making and process for separating them from a product stream in a reactor. A preferred catalyst comprises a catalytically active metal, preferably cobalt, and appropriate promoters, a magnetic support, preferably comprising magnetite, and an encapsulating material, preferably silica, encapsulating the magnetic support.

### TECHNICAL FIELD OF THE INVENTION

The invention generally relates to Fischer-Tropsch catalysts. More specifically, the invention relates to the use of a magnetic support for Fischer-Tropsch catalysts to facilitate the separation of Fischer-Tropsch products from the catalysts. Still more particularly, the invention relates to the encapsulation of magnetite by silica to provide a magnetic support for a cobalt-based Fischer-Tropsch catalyst.

### BACKGROUND OF THE INVENTION

Large quantities of methane, the main component of natural gas, are available in many areas of the world, and natural gas is predicted to outlast oil reserves by a significant margin. However, most natural gas is situated in areas that are geographically remote from population and industrial centers. The costs of compression, transportation, and storage make its use economically unattractive. To improve the economics of natural gas use, much research has focused on the use of methane as a starting material for the production of higher hydrocarbons and hydrocarbon liquids, which are more easily transported and thus more economical. The conversion of methane to hydrocarbons is typically carried out in two steps. In the first step, methane is converted into a mixture of carbon monoxide and hydrogen (i.e., synthesis gas or syngas). In a second step, the syngas is converted into hydrocarbons.

This second step, the preparation of hydrocarbons from synthesis gas, is well known in the art and is usually referred to as Fischer-Tropsch synthesis, the Fischer-Tropsch process, or Fischer-Tropsch reaction(s). Fischer-Tropsch synthesis generally entails contacting a stream of synthesis gas with a catalyst under temperature and pressure conditions that allow the synthesis gas to react and form hydrocarbons.

More specifically, the Fischer-Tropsch reaction is the catalytic hydrogenation of carbon monoxide to produce any of a variety of products ranging from methane to higher alkanes and aliphatic alcohols. Research continues on the development of more efficient Fischer-Tropsch catalyst systems and reaction systems that increase the selectivity for high-value hydrocarbons in the Fischer-Tropsch product stream.

There are continuing efforts to find catalysts that are more effective at producing desired products. Product distribution, product selectivity, and reactor productivity depend heavily on the type and structure of the catalyst and on the reactor type and operating conditions. It is particularly desirable to maximize the production of high-value liquid hydrocarbons, such as hydrocarbons with five or more carbon atoms per hydrocarbon chain (C₅₊).

Catalyst supports for catalysts used in Fischer-Tropsch synthesis of hydrocarbons have typically been oxides (e.g., silica, alumina, titania, zirconia or mixtures thereof, such as silica-alumina). The products prepared by using these catalysts usually have a very wide range of molecular weights. It has been asserted that the Fischer-Tropsch synthesis reaction is only weakly dependent on the chemical identity of the metal oxide support (see E. Iglesia et al. 1993, In: “Computer-Aided Design of Catalysts,” ed. E. R. Becker et al., p. 215, New York, Marcel Dekker, Inc.). Nevertheless, because it continues to be desirable to improve the activity of Fischer-Tropsch catalysts, other types of catalyst supports, including magnetic supports, have been investigated.

Magnetism can be explained as a class of physical phenomena that include the attraction for iron observed in lodestone and a magnet, are inseparably associated with moving electricity, are exhibited by both magnets and electric currents, and are characterized by fields of force. Electrons are perpetually rotating, and, since the electron has a charge, its spin produces a small magnetic moment. Magnetic moments are small magnets with north and south poles. The direction of the moment is from the south to the north pole. In nonmagnetic materials the electron moments cancel, since there is random ordering to the direction of the electron spins. Whenever two electrons have their moments aligned in opposite directions, their effects tend to cancel. Magnets are formed when a large number of the electrons align their individual moments in the same direction. The forces that tend to align the electron spins are subtle. Magnetic is herein defined as susceptible to magnetism.

Iron is a typical ferromagnet. Not all bars of iron are magnets; the existence of magnetism is determined by the nature of the domains within the bar. A domain is a region of a crystal in which all the ions are ferromagnetically aligned in the same direction. A bar may be composed of many domains, each having a different magnetic orientation. Such a bar would not appear to be magnetic. Each piece of the bar is magnetic, but the domains have moments that point in different directions, so the bar has no net moment. If the bar of iron is placed in a strong magnetic field, however, the bar becomes magnetic. The field causes the bar to become a single domain with all moments aligned along the external field. The domains do not rotate their moments; instead, the walls between domains move. The domain with a moment along the field grows, while the others become smaller. If removed from the magnetic field, the iron bar will remain magnetized for a considerable time period. Nearly all bars of iron are polycrystalline: they have many small grains of single crystals, which are packed together with random orientation. A grain could be a single domain, a domain could include many grains, or a large grain could have several domains.

Very small particles (50-350 Angstrom region) of normally ferromagnetic materials are unable to support magnetic domains and are called superparamagnetic. This means that they are weakly magnetic in the absence of an external magnetic field, but upon the application of an external magnetic field, become magnetic and agglomerate readily. The ease with which such particles become magnetized upon application of a magnetic field is directly proportional to their degree of magnetization, measured in emul/gm (electromagnetic units per gram). Their property of becoming demagnetized upon removal of the magnetic field is inversely proportional to their coercive force, measured in Oersteds (Oe). As a practical matter, materials (particles) that have a degree of magnetization of at least about 30 emul/gm and a coercive force of less than about 30 Oe can be considered superparamagnetic. Generally, the greater the magnetization and the lower the coercive force, the more usefully or “strongly” superparamagnetic the particles become. That is, less magnetic force is required to magnetize them and they lose their magnetic properties more rapidly upon removal of the outside magnetic force. Such particles have found many uses, ranging from mechanical seals and couplings to biological separations.

Ferromagnetic materials in general become permanently magnetized in response to magnetic fields. Materials termed “superparamagnetic” experience a force in a magnetic field gradient, but do not become permanently magnetized. Crystals of magnetic iron oxides may be either ferromagnetic or superparamagnetic, depending on the size of the crystals. Superparamagnetic oxides of iron generally result when the crystal is less than about 350 Angstroms in diameter; larger crystals generally have a ferromagnetic character. Following initial exposure to a magnetic field, ferromagnetic particles tend to aggregate because of magnetic attraction between the permanently magnetized particles.

As discussed above, in typical Fischer-Tropsch processes, synthesis gases comprising carbon oxides and hydrogen are reacted in the presence of Fischer-Tropsch catalysts to produce liquid hydrocarbons. Fischer-Tropsch synthesis processes are most commonly conducted in fixed bed, gas-solid or gas-entrained fluidized bed reaction systems, fixed bed reaction systems being the most commonly used. It is recognized in the art, however, that slurry bubble column reactor systems offer tremendous potential benefits over these commonly used Fischer-Tropsch reaction systems. However, the commercial viability of slurry bubble column processes has been questioned. The unique reaction conditions experienced in slurry bubble column processes are extremely harsh. Thus, catalyst attrition losses in slurry bubble column processes can be both very high and costly. In fact, many of the best performing catalysts employed in other Fischer-Tropsch reaction systems quickly break down when used in slurry bubble column systems.

Another problem associated with catalyst use in Fischer-Tropsch synthesis is the separation of catalyst from product in daily operations. As described above, of particular interest is catalyst carried downstream due to poor attrition resistance. Catalyst lost from units caused by poor attrition resistance can be a serious problem, since the quantities lost must be replaced by fresh catalyst additions to maintain constant unit performance. In addition to catalyst loss, the physical destruction and attrition of the catalyst results in (i) poorer distribution of the catalyst in reactors; (ii) filtration problems in removing liquid products; and (iii) possible contamination of products with catalytic material.

As a result, catalyst manufacturers work hard to prevent losses due to attrition, and refiners keep a close watch on catalyst quality to be sure the product conforms to their specifications. Faulty unit operation can also lead to catalyst losses, even with well-made, attrition-resistant catalysts. Hence, it is desired to provide a catalyst that may be easily separated from the product to prevent catalyst loss downstream.

### SUMMARY OF THE INVENTION

The present invention provides a magnetic support for a catalyst, which facilitates the separation of the catalyst from reaction products. While described for use in a Fischer-Tropsch system, the present invention can be extended to other systems wherein silica based catalysts can benefit from enhanced separation schemes.

According to a preferred embodiment, a process for producing hydrocarbons includes contacting a feed stream of hydrogen and carbon monoxide with a catalyst in a reaction zone maintained at conversion-promoting conditions effective to produce an effluent stream of hydrocarbons. The catalyst preferably includes a magnetic support, a catalytically active layer, and an encapsulating layer, which encapsulates the magnetic support. The catalytically active layer preferably comprises a catalytically active metal and promoter, and is preferably supported on the encapsulating layer. The catalytically active metal may be selected from the group including Co, Re, Ni and Ru. Preferably, the catalytically active metal is cobalt. The promoter may be selected from the group including Re, Ru, Rh, Pt, Pd, Ir, Cu, Ag, Zn, V, Cr, Mo, W, Ti, B, Mn, P, Ge, In, Sn, any of the Lanthanide series, and any combinations thereof. The magnetic support preferably comprises magnetite. The encapsulating layer may be selected from the group including silica, alumina, titania, and any combinations thereof. Preferably, the encapsulating layer comprises silica. In some embodiments, the catalyst may be pretreated with hydrogen.

According to an alternate preferred embodiment, a silica-supported catalyst includes a magnetic support, a catalytically active layer, and a silica layer, which encapsulates the magnetic support. In some embodiments, the silica-supported catalyst is a Fischer-Tropsch catalyst.

According to still another preferred embodiment, a method for separating a catalyst in a catalyst bed from a hydrocarbon product stream includes running a Fischer-Tropsch reaction and applying a magnetic field over the catalyst bed, wherein the catalyst includes a magnetic support, a catalytically active layer, and an encapsulating layer, which encapsulates the magnetic support.

While the above catalysts have been described in terms of “layers”, it should be understood that the layers may be separate and distinct or coexist in a single layer. Other objects and advantages of the present invention will appear from the following description.

### BRIEF DESCRIPTION OF THE DRAWINGS

A better understanding of the present invention can be obtained when the following detailed description of the preferred embodiment is considered in conjunction with the following drawings:

FIG. 1 is a schematic drawing of a catalyst particle in accordance with a preferred embodiment of the present invention;

FIGS. 2A, 2B are schematic drawings of a catalytic system in accordance with a preferred embodiment of the present invention;

FIGS. 3A, 3B are transmission electron microscope (TEM) images showing morphology of precipitated iron oxide before (3A) and after (3B) encapsulation with Ludox® AS silica;

FIG. 4 shows XRD powder patterns of precipitated monodispersed crystalline iron oxides;

FIGS. 5A, 5B are TEM images of precipitated monodispersed crystalline hematite spindles;

FIGS. 6A, 6B are TEM images of precipitated monodispersed crystalline akaganeite;

FIG. 7 shows XRD powder patterns of precipitated monodisperse iron oxides after a reduction treatment; and

FIGS. 8A, 8B are TEM images of precipitated monodisperse iron oxides after a reduction treatment.

### DETAILED DESCRIPTION OF THE PREFERRED EMBODIMENTS

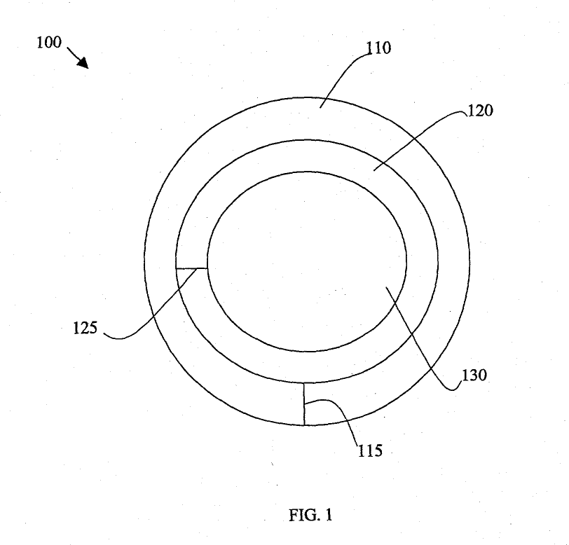

Referring initially to FIG. 1, one embodiment of the present system, a catalyst particle 100 in a preferred catalyst system includes an encapsulating layer 120, a catalytically active layer 110, and a magnetic support 130. Encapsulating layer 120 is preferably comprised of an oxide such as silica, alumina, titania, zirconia, barium oxide, lanthanum oxide, thoria, and any combinations thereof, and has a thickness 125 of approximately 1 nm to 5 microns. In a preferred embodiment, encapsulating layer 120 is selected from the group including silica, alumina, titania, zirconia and any combinations thereof. Preferably, encapsulating layer 120 is silica. Catalytically active layer 110 is preferably comprised of a catalytically active metal selected from the group including Co, Re, Ni and Ru and a promoter selected from the group including Re, Ru, Rh, Pt, Pd, Ir, Cu, Ag, Zn, V, Cr, Mo, W, Ti, B, Mn, P, Ge, In, Sn, any of the Lanthanide series, and any combinations thereof Catalytically active layer 110 preferably has a thickness 115 of approximately 1 nm to 5 microns. Preferably, the catalytically active metal is cobalt. Magnetic support 130 is preferably paramagnetic. In a preferred embodiment, magnetic support 130 is comprised of magnetite.

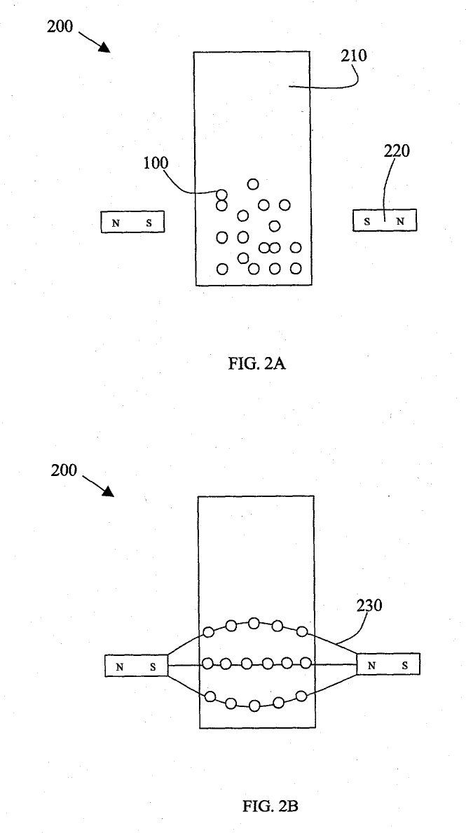

Referring now to FIG. 2A, a catalytic system 200 is shown, including a reactor 210, electromagnets 220, and catalyst particles 100. In catalytic system 200, reactants (not shown) pass over catalyst particles 100, forming products (not shown). In this first stage, electromagnets 220 are off, allowing catalyst particles 100 to move freely in reactor 210. As products accumulate, it is desirable to remove them from reactor 210.

When enough products accumulate, catalyst particles 100 may be separated from the products by suspending the catalyst in a magnetic field. Referring now to FIG. 2B, electromagnets 220 apply a magnetic field 230 to catalyst particles 100, forcing the magnetic domains (not shown) in magnetic support 130 to align. Following initial exposure to the magnetic field, catalyst particles 100 tend to aggregate because of the magnetic attraction between the magnetized catalyst particles. The magnetic field retains the aggregated catalyst particles in reactor 210 as long as it is applied. Products such as hydrocarbons are allowed to exit the reactor essentially catalyst free. Once a sufficient amount of products are removed from reactor 210, electromagnets 210 are turned off, and catalytic system 200 returns to the first stage shown in FIG. 2A.

By use of electromagnets to supply a magnetic field, the catalyst will be separated by attraction to the magnet and allow the product to pass on. Encapsulation of magnetite by silica will supply the magnetic support for cobalt Fischer-Tropsch catalysts. Once the encapsulated particle is produced, cobalt and appropriate promoters can be supported on the magnetic support via standard methods (i.e. incipient wetness wet impregnation). Catalytic performance of the resulting catalyst will be comparable to conventional cobalt Fischer-Tropsch catalyst supported on silica.

The resulting catalyst may comprise powders, particles, pellets, granules, spheres, beads, pills, balls, noodles, cylinders, extrudates, trilobes, monoliths, honeycombs, packed beds, foams, and aerogels. The terms “distinct” or “discrete” structures or units, as used herein, refer to supports in the form of divided materials such as granules, beads, pills, pellets, cylinders, trilobes, extrudates, spheres or other rounded shapes, or another manufactured configuration. Alternatively, the divided material may be in the form of irregularly shaped particles or particulates.

A further advantage to the present invention is that the encapsulation prevents the magnetite support from contacting the reaction mixture. It is important for the magnetite support to be encapsulated, because magnetite is known to be a high temperature (temperatures in excess of 350° C.) catalyst that promotes the water gas shift (WGS, Equation 1).

CO+H₂O→CO₂+H₂ (1)

As shown in Equation (1), WGS increases the carbon dioxide yield and lowers the carbon efficiency of the whole Fischer-Tropsch process. The silica coating prevents the reaction mixture of the Fischer-Tropsch synthesis from reaching the iron oxide, thus minimizing the water gas shift, and subsequently maximizing carbon efficiency.

### Support Preparation

Hematite Production

An iron nitrate solution was raised to a pH of 4.5 by the addition of NH₄OH under continuous stirring. After 30 minutes, the resulting suspension was centrifuged to recover the precipitate. XRD analysis of the precipitate (dried at 100° C.) confirmed the identity as hematite.

An alternate preparation can be performed by adjusting the pH to 8.4.

Magnetite Production

Magnetite was produced by reduction of precipitated iron oxides, which can be, but are not limited to, hematite, amorphous iron oxide, monodisperse hematite, and monodisperse akaganeite. Hematite was utilized for demonstration of this invention.

Encapsulated Magnetite Production

Encapsulated magnetite can be produced by encapsulation of magnetite particles with a layer of silica. This is accomplished via several chemical treatment procedures to the magnetite, including coating magnetite with SiO₂ using a sol gel technique or treatment with silicic acid (H₄SiO₄) or commercial silica sol, such as Ludox®.

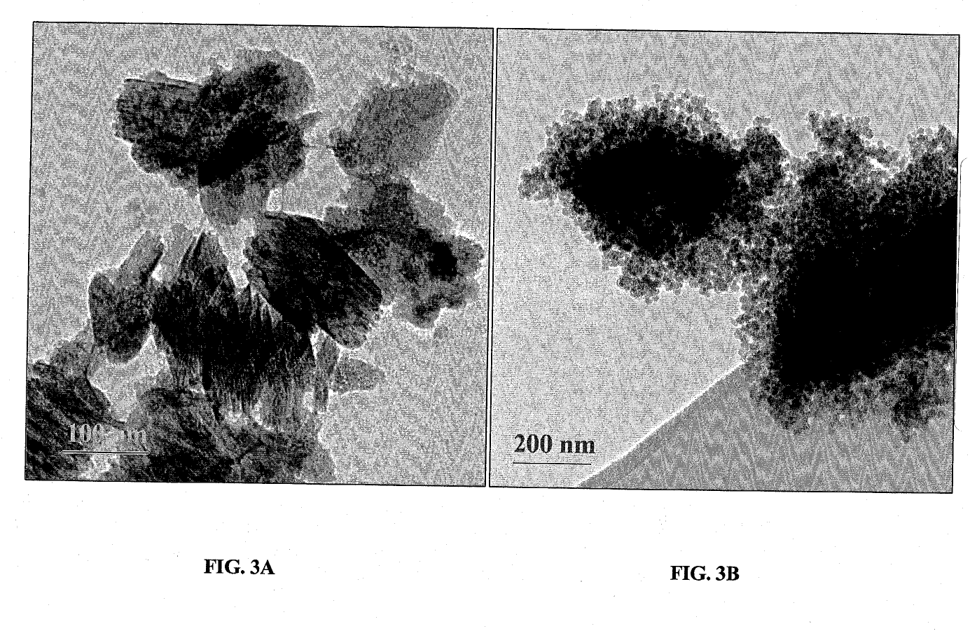

Referring now to FIGS. 3A, 3B, TEM images showing morphology of precipitated amorphous iron oxide before and after encapsulation with Ludox®) AS silica are shown. Before encapsulation (FIG. 3A), the primary size of precipitated hematite particles is in the range of 50-150 nm. As can be seen, in FIG. 3B, hematite particles are perfectly coated with a thick layer of silica after the controlled encapsulation treatment.

Monodispersed Crystalline Hematite Production

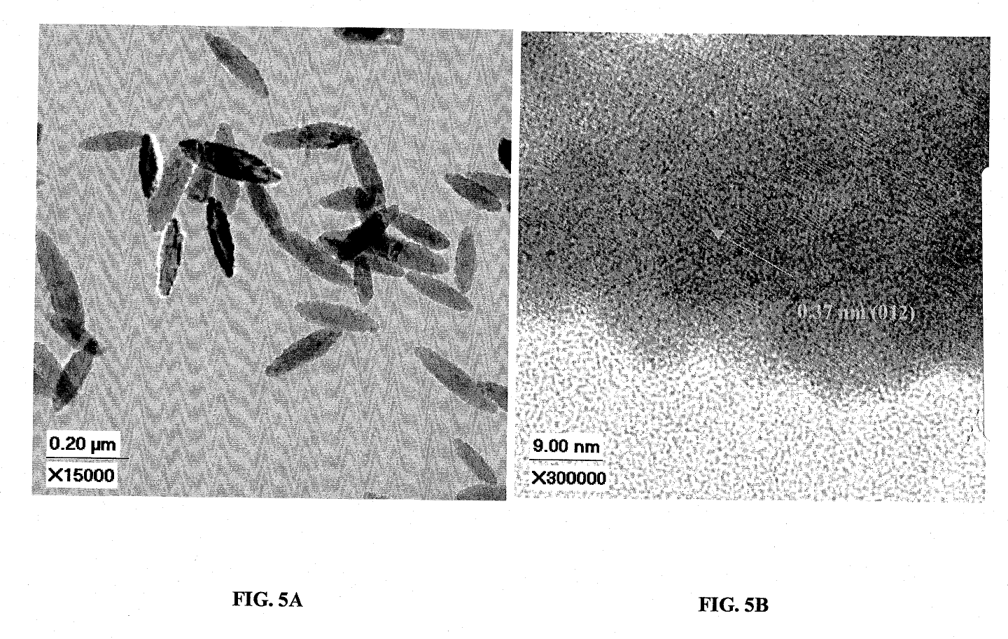

An aqueous solution containing 0.02 M FeCl₃ and 0.0003 M Na₃PO₄ was aged in a tightly stoppered Pyrex flask in a pre-heated oven at 100° C. for 4 days. The suspension was centrifuged and washed with deionized water. The resultant iron oxide was dried in an oven at 110° C. overnight. XRD showed that the formed iron oxide was hematite. The average crystal size calculated through XRD measurement is 39 nm. The low magnification TEM image in FIG. 5A shows the unique spindle morphology of the precipitate with uniform particle size of about 30 nm×100 nm. The high resolution TEM image in FIG. 5B shows that each spindle of the precipitate is a hematite single crystal.

The powder form of the precipitated monodispersed hematite crystals was then reduced in a quartz tube furnace under a hydrogen flow for 1 hour at a temperature of 350° C.

Monodispersed Crystalline Akaganeite Production

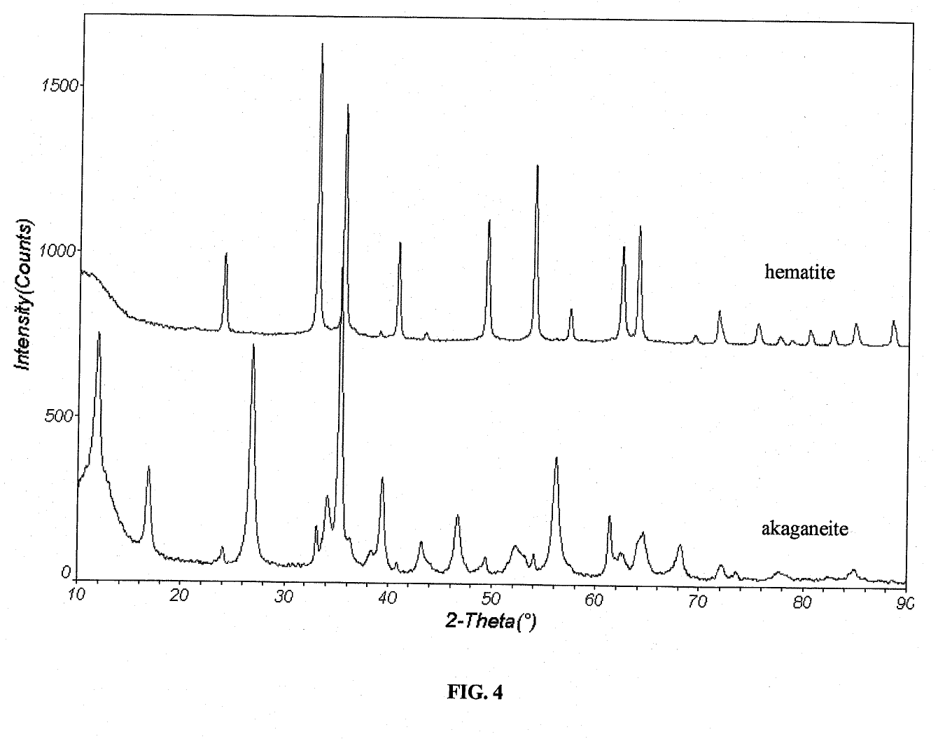

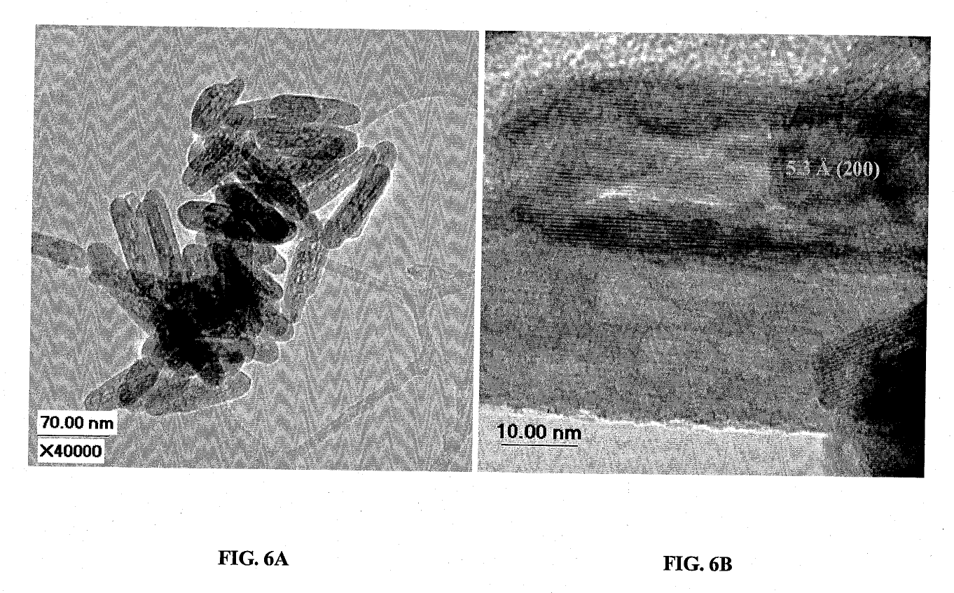

An iron nitrate solution was adjusted to a pH of 10.7 by the slow addition of aqueous KOH. The precipitated suspension was agitated overnight at room temperature and washed 4 times with deionized water. The washed iron oxide precipitate was then resuspended in 1 L of water and buffered by the addition of 30 ml 1 M HCl and 1.5 ml 0.1 M Na₃PO₄ to the suspension. The suspension was aged in a tightly stoppered Pyrex flask in a pre-heated oven at 100° C. for 4 days. The suspension was centrifuged and washed with deionized water. The resultant iron oxide was dried in an oven at 110° C. overnight. XRD showed that the formed iron oxide was akaganeite (see FIG. 4). The average crystal size of akaganeite calculated through XRD measurement is 14.7 nm. Referring now to FIG. 6A, TEM measurement shows that the precipitated akageneite particles have a unique peanut shape with a size of about 20 nm (width)×80 nm (length). The high resolution TEM image in FIG. 6B shows each peanut shaped akaganeite particle is a single crystal.

The powder form of the precipitated monodispersed akaganeite was then reduced in a quartz tube furnace under a hydrogen flow for 1 hour at a temperature of 350° C.

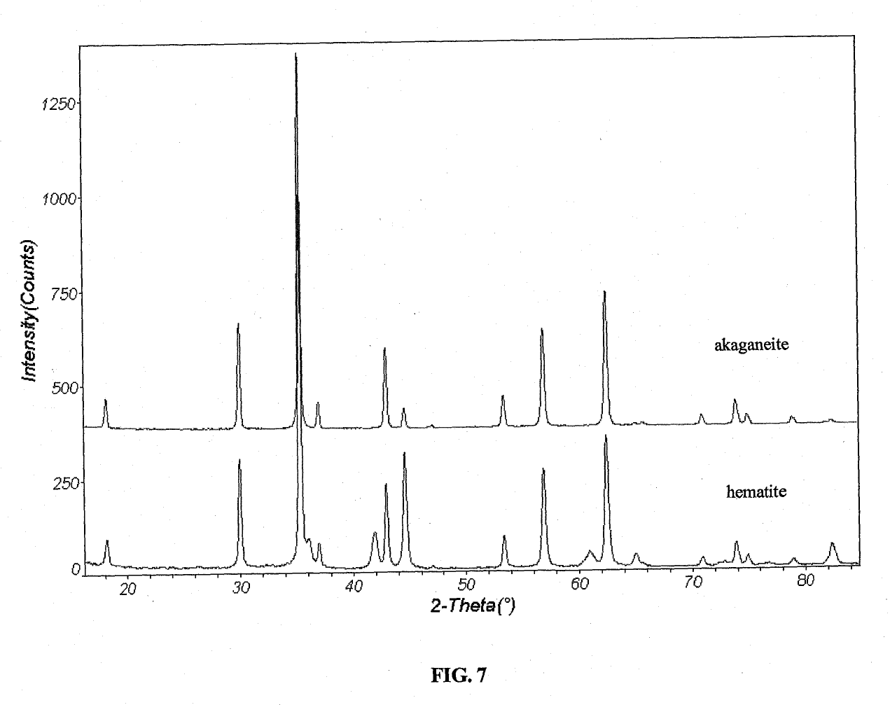

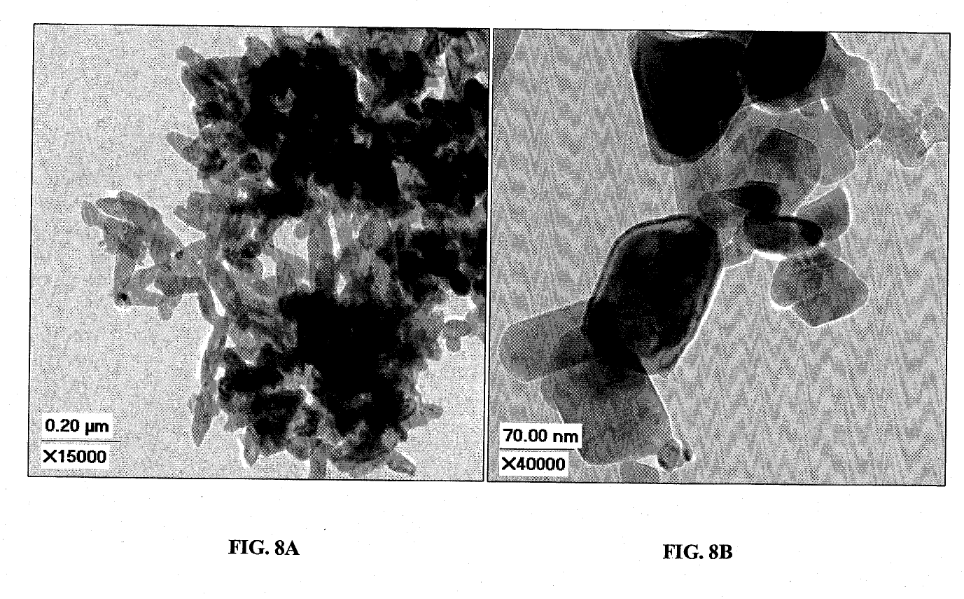

Referring now to FIGS. 7 and 8, XRD graphs and TEM images of magnetite, produced from monodispersed iron oxide precursors (hematite and akaganeite) are shown. XRD graphs in FIG. 7 show that after a reduction treatment with hydrogen at 350° C., both hematite and akaganeite were transformed to magnetite. In FIG. 7, it is also shown that a minor amount of metallic iron was also formed, especially for the sample of hematite precursor. The low magnification TEM image in FIG. 8A shows that the resultant magnetite particles from the precipitated hematite spindles still keep the spindle shape with a size similar to that of the precursor. Conversely, the TEM image in FIG. 8B shows that the resultant magnetite particles from the monodisperse akaganeite precursor lose the uniform peanut shape morphology. A comparison of the size of magnetite particles and that of the akaganeite precursor particles indicates sintering of the resultant magnetite. Further controlled reduction can be performed to produce monodisperse magnetite.

Technique I: Encapsulation of Monodisperse Magnetite Particles by Commercial Silica Sol

Magnetite powder from the reduction of precipitated iron oxides was ground and re-dispersed in DI water to form a suspension. The suspension was further sonicated to ensure complete dispersion of the magnetite in water. The pH of the solution was adjusted to between 4 and 5 with dilute nitric acid. With continuous and vigorous stirring, Ludox® AS30 silica sol was added in a drop wise fashion to this magnetite suspension while keeping the pH between 4 and 5 by further addition of nitric acid. Stirring was continued for 0.5 h after the addition of the silica sol. The resulting product was dried at 110° C. after filtering. XRD measurement showed that the encapsulated iron oxide remained magnetite. The amount of silica sol added can be varied depending on the thickness of the encapsulating silica layer.

Technique II: Encapsulation of Monodisperse Magnetite Particles by a Sol-Gel Method

Magnetite produced from reduction of precipitated monodisperse iron oxide was suspended in ammoniacal ethanol. This suspension was stirred and sonicated for 0.5 h to help disperse magnetite particles. Tetraethylorthosilicate (TEOS) was quickly added to the suspension under vigorous stirring. This suspension was then aged at 40° C. overnight. After centrifugation the resulting solid was dried at 110° C. XRD measurement showed that this encapsulated iron oxide is magnetite.

The silica-encapsulated magnetite powder produced by either Technique I or II can be used as a catalytic support material to prepare a supported cobalt catalyst by a conventional impregnation method. The cobalt catalysts supported on silica-encapsulated magnetite are preferably reduced with hydrogen at a temperature of at least 400° C. before use as a Fischer-Tropsch catalyst.

### Operation

The present catalysts are preferably used in a Fischer-Tropsch reactor charged with feed gases comprising hydrogen or a hydrogen source and carbon monoxide. H₂/CO mixtures suitable as a feedstock for conversion to hydrocarbons according to the process of this invention can be obtained from light hydrocarbons such as methane by means of steam reforming, autothermal reforming, or partial oxidation. The hydrogen is preferably provided by free hydrogen, although some Fischer-Tropsch catalysts have sufficient water gas shift activity to convert some water to hydrogen for use in the Fischer-Tropsch process. It is preferred that the mole ratio of hydrogen to carbon monoxide in the feed be greater than 0.5:1 (e.g., from about 0.67:1 to 2.5:1). The feed gas may also contain carbon dioxide or other compounds that are inert under Fischer-Tropsch reaction conditions, including but not limited to nitrogen, argon, or light hydrocarbons. The feed gas stream should contain a low concentration of compounds or elements that have a deleterious effect on the catalyst. The feed gas may need to be treated to ensure low concentrations of sulfur or nitrogen compounds such as hydrogen sulfide, ammonia and carbonyl sulfides.

The feed gas is contacted with the catalyst in a reaction zone. Mechanical arrangements of conventional design may be employed as the reaction zone. For example, fixed bed, slurry phase, slurry bubble column, fluidized bed, or ebulliating bed reactors. Accordingly, the size of the catalyst particles may vary depending on the reactor in which they are to be used.

The process is typically run in a continuous mode. In this mode, typically, the gas hourly space velocity through the reaction zone may range from about 100 volumes/hour/volume catalyst (v/hr/v) to about 10,000 v/hr/v, preferably from about 300 v/hr/v to about 2,000 v/hr/v. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psig (653 kPa) to about 1000 psig (6994 kPa), preferably, from 80 psig (653 kPa) to about 600 psig (4237 kPa), more preferably, from about 140 psig (1066 kPa) to about 400 psig (2858 kPa).

The reaction products will have a large range of molecular weights. The present catalysts are particularly useful for making hydrocarbons having five or more carbon atoms, especially when the above-referenced space velocity, temperature and pressure ranges are employed.

The wide range of hydrocarbon species produced in the reaction zone will typically result in liquid phase products at the reaction zone operating conditions. Therefore, the effluent stream of the reaction zone will often be a mixed phase stream. The effluent stream of the reaction zone may be cooled to effect the condensation of additional amounts of hydrocarbons and passed into a vapor-liquid separation zone. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column wherein they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following embodiments are to be construed as illustrative, and not as constraining the remainder of the disclosure in any way whatsoever. For example, while the invention has been described for use in a Fischer-Tropsch process, it can be translated to any silica-supported catalyst.

What is claimed is:

1. A process for producing hydrocarbons, comprising contacting a feed stream comprising hydrogen and carbon monoxide with a catalyst in a reaction zone maintained at conversion-promoting conditions effective to produce an effluent stream comprising hydrocarbons, wherein the catalyst comprises:
a magnetic support;
an encapsulating layer; and
a catalytically active layer;
wherein the encapsulating layer encapsulates the magnetic support and wherein the catalytically active layer is disposed on the encapsulating layer.

2 The process according to claim 1 wherein the catalytically active layer comprises a catalytically active metal and a promoter.

3. The process according to claim 2 wherein the catalytically active metal is selected from the group consisting of Co, Re, Ni, Fe and Ru.

4. The process according to claim 3 wherein said catalytically active metal is essentially cobalt.

5. The process according to claim 2 wherein said promoter is selected from the group consisting of Re, Ru, Rh, Pt, Pd, Ir, Cu, Ag, Zn, V, Cr, Mo, W, Ti, B, Mn, P, Ge, In, Sn, any of the Lanthanide series, and any combinations thereof.

6. The process according to claim 1 wherein the catalytically active layer is approximately 10 nm to 200 microns thick.

7. The process according to claim 1 wherein the catalyst is comprised of a plurality of discrete structures.

8. The process according to claim 7 wherein the discrete structures are particulates.

9. The process according to claim 7 wherein the plurality of discrete structures comprises at least one geometry chosen from the group consisting of powders, particles, pellets, granules, spheres, beads, pills, balls, noodles, cylinders, extrudates and trilobes.

10. The process according to claim 1 wherein the magnetic support is paramagnetic.

11. The process according to claim 1 wherein the magnetic support comprises magnetite.

12. The process according to claim 11 wherein the magnetite is produced from an amorphous iron oxide precursor.

13. The process according to claim 11 wherein the magnetite is produced from a crystalline hematite precursor.

14. The process according to claim 11 wherein the magnetite is produced from a crystalline akaganeite precursor.

15. The process according to claim 1 wherein the encapsulating layer comprises an oxide.

16. The process according to claim 15 wherein the encapsulating layer comprises an oxide selected from the group consisting of silica, alumina, titania, and any combinations thereof.

17. The process according to claim 16 wherein the encapsulating layer comprises silica.

18. The process according to claim 1 wherein the encapsulating layer is approximately 5 nm to 200 microns thick.

19. The process according to claim 1 wherein the catalyst is pretreated with hydrogen.

20. A silica supported catalyst comprising:
a magnetic support;
a silica-comprising layer; and
a catalytically active layer;
wherein the silica-comprising layer encapsulates the magnetic support and wherein the catalytically active layer is disposed on the silica-comprising layer.

21. A Fischer-Tropsch catalyst comprising:
a magnetic support;
an encapsulating layer; and
a catalytically active layer;
wherein the encapsulating layer encapsulates the magnetic support and wherein the catalytically active layer is disposed on the encapsulating layer.

22. A method for preparing a Fischer-Tropsch catalyst comprising:
providing a magnetic support;
providing an encapsulating layer; and
providing a catalytically active layer;
wherein the encapsulating layer encapsulates the magnetic support and wherein the catalytically active layer is disposed on the encapsulating layer.

23. The method according to claim 22 wherein the magnetic support is produced by precipitating and reducing an amorphous iron oxide precursor.

24. The method according to claim 23 wherein the encapsulating layer is produced using a silica sol precursor.

25. The method according to claim 23 wherein the encapsulating layer is produced using a sol gel precursor.

26. The method according to claim 24 wherein the catalytically active layer is disposed on the encapsulating layer by an incipient wetness technique.

27. The method according to claim 25 wherein the catalytically active layer is disposed on the encapsulating layer by an impregnation technique.

28. The method according to claim 22 wherein the magnetic support is produced by precipitating and reducing a crystalline hematite precursor.

29. The method according to claim 22 wherein the magnetic support is produced by precipitating and reducing a crystalline akaganeite precursor.

30. A method for separating a catalyst in a catalyst bed from a hydrocarbon product stream comprises running a Fischer-Tropsch reaction and applying a magnetic field over the catalyst bed, wherein the catalyst comprises a magnetic support, an encapsulating layer, and a catalytically active layer, wherein the encapsulating layer encapsulates the magnetic support and wherein the catalytically active layer is disposed on the encapsulating layer.
